(19) United States
(12) Patent Application Publication
(10) Pub. No.: US 2025/0009513 A1
(43) Pub. Date: Jan. 9, 2025

(54) MICRO-FLUIDIC DEVICE COMBINING FUNCTIONS OF A PUMP AND A VALVE

(71) Applicant: Boston Scientific Scimed, Inc., Maple Grove, MN (US)

(72) Inventors: Martin Carey, Clonmel (IE); Noel Smith, Windgap (IE); Eduardo Marcos Larangeira, Cork City (IE); Thomas Sinnott, Wexford (IE); Richard Percy, Leamlara (IE); Daragh Nolan, Via Youghal (IE)

(21) Appl. No.: 18/760,646

(22) Filed: Jul. 1, 2024

Related U.S. Application Data
(60) Provisional application No. 63/512,141, filed on Jul. 6, 2023.

(51) Int. Cl. A61F 2/26; A61F 2/48

(52) U.S. Cl. CPC A61F 2/26; A61F 2/484; A61F 2250/0013

(57) ABSTRACT

An implantable fluid operated device may include a fluid reservoir configured to hold fluid, an inflatable member, and an electronic fluid control system to transfer fluid between the fluid reservoir and the inflatable member. The fluid control system includes at least one combination pump and valve device including a piezoelectric actuator.

### CROSS-REFERENCE TO RELATED APPLICATION

This application claims priority to U.S. Provisional Patent Application No. 63/512,141, filed on Jul. 6, 2023, entitled “MICRO-FLUIDIC DEVICE COMBINING FUNCTIONS OF A PUMP AND A VALVE”, the disclosure of which is incorporated by reference herein in its entirety.

### TECHNICAL FIELD

This disclosure relates generally to bodily implants, and more specifically to bodily implants including a fluid control system having one or more pumps and/or valves including a piezoelectric actuator.

### BACKGROUND

Active implantable fluid operated inflatable devices can include one or more pumps that regulate a flow of fluid between different portions of the implantable device. One or more valves can be positioned within fluid passageways of the device to direct and control the flow of fluid to achieve inflation, deflation, pressurization, depressurization, activation, deactivation and the like of different fluid filled implant components of the device. In some implantable fluid operated devices, an implantable pumping device may be manually operated by the user to provide for the transfer of fluid between a reservoir and the fluid filled implant components of the device. Manipulation of the manually operated implantable pumping device may be challenging for some patients. Further, such manual operation of the pumping device may make it may be difficult to achieve consistent inflation, deflation, pressurization, depressurization, activation, deactivation and the like of the fluid filled implant components. Inconsistent inflation, deflation, pressurization, depressurization, activation and/or deactivation of the fluid filled implant device(s) may adversely affect patient comfort, efficacy of the device, and the overall patient experience. Some implantable fluid operated devices may include an electronic control system including an electronically controlled manifold providing for the transfer of fluid within the implantable fluid operated device. The use of the electronic control system may provide for more accurate actuation and control of the flow of fluid between components of the inflatable device, thus improving performance and efficacy of the device, as well as patient comfort and safety. In some situations, a size and/or a configuration of the electronic control system, driven at least in part by the number and/or positioning pumps and/or valves within a manifold of the electronic control system, may pose a challenge to some patients.

### SUMMARY

In a general aspect, an implantable fluid operated inflatable device includes a fluid reservoir; an inflatable member; and a fluid control system configured to control fluid flow between the fluid reservoir and the inflatable member. The fluid control system may include fluidic architecture defining one or more fluid passageways within in a housing; and at least one combined pump and valve device positioned in the one or more fluid passageways, the at least one combined pump and valve device. The at least one combined pump and valve device may include a base plate; a passive foil layer positioned in a recessed portion of the base plate; an actuator plate coupled to the base plate so as to define a chamber with the recessed portion of the base plate; a first disc selectively engaging a first opening formed in a first valve pocket in the base plate or a first opening in the passive foil layer in response to a voltage applied to the actuator plate; and a second disc selectively engaging a second opening formed in a second valve pocket in the base plate or a second opening in the passive foil layer in response to a voltage applied to the actuator plate.

In some implementations, the first opening in the base plate has a substantially circular shape, the first opening in the passive foil layer has a substantially elliptical shape, the second opening in the base plate has a substantially elliptical shape, and the second opening in the passive foil layer has a substantially circular shape.

In some implementations, in response to a first voltage applied to the actuator plate, the first disc is positioned against the first opening in the passive foil layer, and the second disc is positioned against the second opening in the passive foil layer. In response to a second voltage applied to the actuator plate, the first disc is positioned against the first opening in the base plate, and the second disc is positioned against the second opening in the base plate. In response to the first voltage applied to the actuator plate, the first disc partially blocks the first opening in the passive foil layer; and the second disc fully blocks the second opening in the passive foil layer. In response to the first voltage applied to the actuator plate, fluid flows into the chamber through the first opening formed in the base plate and through open end portions of the first opening in the passive foil layer; and fluid is retained in the chamber by the second disc positioned against the second opening in the passive foil layer to fill the chamber with fluid. In response to the second voltage applied to the actuator plate, the first disc fully blocks the first opening in the base plate; and the second disc partially blocks the second opening in the base plate. In response to the second voltage applied to the actuator plate, fluid flows out of the chamber through open end portions of the second opening formed in the passive foil layer and through the second opening in the base plate; and backflow of fluid from the chamber is restricted by the first disc positioned against the first opening in the base plate.

In some implementations, the base plate includes a base portion; and a rim portion extending along a peripheral portion of the base portion. The recessed portion of the base plate may be formed within an area defined by the base portion and the rim portion. The first valve pocket and the second valve pocket may be formed in the base portion of the base plate.

In some implementations, the at least one combined pump and valve device includes a seal provided on an interior facing side of the actuator plate, wherein the seal extends down into recessed portion of the base plate to seal the chamber.

In some implementations, the first disc is a floating disc positioned within the first valve pocket formed in the base plate, such that the first disc is movable in the first valve pocket, between the first opening in the base plate and the first opening in the passive foil layer, in response to an application of voltage to the actuator plate; and the second disc is a floating disc positioned within the second valve pocket formed in the base plate, such that the second disc is movable in the second valve pocket, between the second opening in the base plate and the second opening in the passive foil layer, in response to an application of voltage to the actuator plate.

In some implementations, the actuator plate is operable in response to an application of voltage thereto by an electronic control system of the fluid control system. In some implementations, the actuator plate comprises a piezoelectric actuator that is operable in response to an application of voltage thereto by an electronic control system of the fluid control system.

In some implementations, the first valve pocket, the first opening in the base plate, the first disc, and the first opening in the passive foil layer define a first pocket valve controlling a flow of fluid from a first fluid passageway into the chamber; and the second valve pocket, the second opening in the base plate, the second disc, and the second opening in the passive foil layer define a second pocket valve controlling a flow of fluid out of the chamber to at least one second fluid passageway. The first fluid passageway may provide for fluid communication between the fluid reservoir and the fluid control system, and the at least one second fluid passageway may provide for fluid communication between inflatable member and the fluid control system.

In another general aspect, an implantable fluid operated inflatable device includes a fluid reservoir; an inflatable member; and a fluid control system configured to control fluid flow between the fluid reservoir and the inflatable member. The fluid control system may include at least one combined pump and valve device positioned in one or more fluid passageways defined within a housing. The at least one combined pump and valve device may include a base plate; a passive foil layer positioned in a recessed portion of the base plate; an actuator plate coupled to the base plate so as to define a chamber with the recessed portion of the base plate; a first disc selectively engaging a first opening formed in a first valve pocket in the base plate or a first opening in the passive foil layer in response to a voltage applied to the actuator plate; and a second disc selectively engaging a second opening formed in a second valve pocket in the base plate or a second opening in the passive foil layer in response to a voltage applied to the actuator plate.

In some implementations, the first opening in the base plate has a substantially circular shape, the first opening in the passive foil layer has a substantially elliptical shape, the second opening in the base plate has a substantially elliptical shape, and the second opening in the passive foil layer has a substantially circular shape.

In some implementations, in response to a first voltage applied to the actuator plate, the first disc is positioned against the first opening in the passive foil layer, and the second disc is positioned against the second opening in the passive foil layer, and in response to a second voltage applied to the actuator plate, the first disc is positioned against the first opening in the base plate, and the second disc is positioned against the second opening in the base plate. In response to the first voltage applied to the actuator plate, the first disc partially blocks the first opening in the passive foil layer; and the second disc fully blocks the second opening in the passive foil layer. In response to the first voltage applied to the actuator plate, fluid flows into the chamber through the first opening formed in the base plate and through open end portions of the first opening in the passive foil layer; and fluid is retained in the chamber by the second disc positioned against the second opening in the passive foil layer to fill the chamber with fluid. In response to the second voltage applied to the actuator plate, the first disc fully blocks the first opening in the base plate; and the second disc partially blocks the second opening in the base plate. In response to the second voltage applied to the actuator plate, fluid flows out of the chamber through open end portions of the second opening formed in the passive foil layer and through the second opening in the base plate; and backflow of fluid from the chamber is restricted by the first disc positioned against the first opening in the base plate.

In some implementations, the base plate includes a base portion; and a rim portion extending along a peripheral portion of the base portion. The recessed portion of the base plate may be formed within an area defined by the base portion and the rim portion, and the first valve pocket and the second valve pocket may be formed in the base portion of the base plate.

In some implementations, the at least one combined pump and valve device includes a seal provided on an interior facing side of the actuator plate, wherein the seal extends down into recessed portion of the base plate to seal the chamber.

In some implementations, the first disc is a floating disc positioned within the first valve pocket formed in the base plate, such that the first disc is movable in the first valve pocket, between the first opening in the base plate and the first opening in the passive foil layer, in response to an application of voltage to the actuator plate; and the second disc is a floating disc positioned within the second valve pocket formed in the base plate, such that the second disc is movable in the second valve pocket, between the second opening in the base plate and the second opening in the passive foil layer, in response to an application of voltage to the actuator plate.

In some implementations, the actuator plate is operable in response to an application of voltage thereto by an electronic control system of the fluid control system. In some implementations, the actuator plate comprises a piezoelectric actuator.

In some implementations, the first valve pocket, the first opening in the base plate, the first disc, and the first opening in the passive foil layer define a first pocket valve controlling a flow of fluid from a first fluid passageway into the chamber; and the second valve pocket, the second opening in the base plate, the second disc, and the second opening in the passive foil layer define a second pocket valve controlling a flow of fluid out of the chamber to at least one second fluid passageway.

In some implementations, the first fluid passageway provides for fluid communication between the fluid reservoir and the fluid control system, and the at least one second fluid passageway provides for fluid communication between inflatable member and the fluid control system.

In some implementations, the base plate is machined into a surface of the housing of the fluid control system, or fabricated as a separate unit from the housing of the fluid control system.

In another general aspect, a fluid control system for an implantable fluid operated inflatable device includes a housing; fluidic architecture defining one or more fluid passageways within the housing; and at least one combined pump and valve device positioned a fluid passageway of the fluid control system. The at least one combined pump and valve device may include a base plate; an actuator plate coupled to a rim portion of the base plate so as to define a chamber with a recessed portion of the base plate; a passive foil layer positioned between the base plate and the actuator plate; a first disc selectively engaging a first opening formed in a first valve pocket in the base plate or a first opening in the passive foil layer in response to a voltage applied to the actuator plate; and a second disc selectively engaging a second opening formed in a second valve pocket in the base plate or a second opening in the passive foil layer in response to a voltage applied to the actuator plate. An open area of the first opening in the passive foil layer may extend beyond an outer periphery of the first disc. An open area of the second opening in the base plate may extend beyond an outer periphery of the second disc.

In some implementations, in response to a first voltage applied to the actuator plate, the first disc is positioned against the first opening in the passive foil layer, and the second disc is positioned against the second opening in the passive foil layer, and in response to a second voltage applied to the actuator plate, the first disc is positioned against the first opening in the base plate, and the second disc is positioned against the second opening in the base plate.

In some implementations, in response to the first voltage applied to the actuator plate, the first disc partially blocks the first opening in the passive foil layer, allowing fluid to into the chamber through the first opening formed in the base plate and through open end portions of the first opening in the passive foil layer; and the second disc fully blocks the second opening in the passive foil layer, retaining fluid to fill the chamber with fluid.

In some implementations, in response to the second voltage applied to the actuator plate, the second disc partially blocks the second opening in the base plate, allowing fluid to flow out of the chamber through open end portions of the second opening formed in the passive foil layer and through the second opening in the base plate; and the first disc fully blocks the first opening in the base plate, restricting backflow of fluid from the chamber.

In some implementations, a seal is provided on an interior facing side of the actuator plate. The seal may extend down into the chamber to seal the chamber.

### BRIEF DESCRIPTION OF THE DRAWINGS

FIG. 1 is a block diagram of an implantable fluid operated inflatable device according to an aspect.

FIG. 2 illustrates a system including an example implantable fluid operated inflatable device according to an aspect.

FIG. 3 is a schematic diagram of a fluidic architecture of an implantable fluid operated inflatable device according to an aspect.

FIG. 4A is an exploded view of an example combined pump and valve device of a fluid control system of a fluid operated inflatable device according to an aspect,

FIG. 4B is a perspective view of an example base plate of the example combined pump and valve device shown in FIG. 4A.

FIG. 4C is a perspective view of example floating disks positioned on the example base plate shown in FIG. 4B.

FIG. 4D is a perspective view of an example passive foil layer positioned on the example base plate and floating disks shown in FIG. 4C.

FIG. 4E is a perspective view of an example seal positioned on the example base plate, floating discs, and passive foil shown in FIG. 4D.

FIG. 4F is an assembled view of the example combined pump and valve device shown in FIG. 4A.

FIG. 5 is a cross-sectional view taken along line A-A of FIG. 4E.

FIGS. 6A and 6B are cross-sectional views taken along line A-A of FIG. 4E, illustrating illustrate operation of an example combined pump and valve device, according to an aspect.

FIG. 7A is an exploded view of an example combined pump and valve device of a fluid control system of a fluid operated inflatable device according to an aspect.

FIG. 7B is a perspective view of an example base plate of the example combined pump and valve device shown in FIG. 7A.

FIG. 7C is a perspective view of an example first passive foil layer positioned on the example base plate shown in FIG. 7B.

FIG. 7D is a perspective view of an example second passive foil layer positioned on the example first passive foil layer and base plate shown in FIG. 7C.

FIG. 7E is a perspective view of an example seal positioned on the example base plate, first passive foil layer, and second passive foil layer shown in FIG. 7D.

FIG. 7F is an assembled view of the example combined pump and valve device shown in FIG. 7A.

FIGS. 8A and 8B are cross-sectional views taken along line B-B of FIG. 7F, illustrating operation of an example combined pump and valve device, according to an aspect.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having” as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

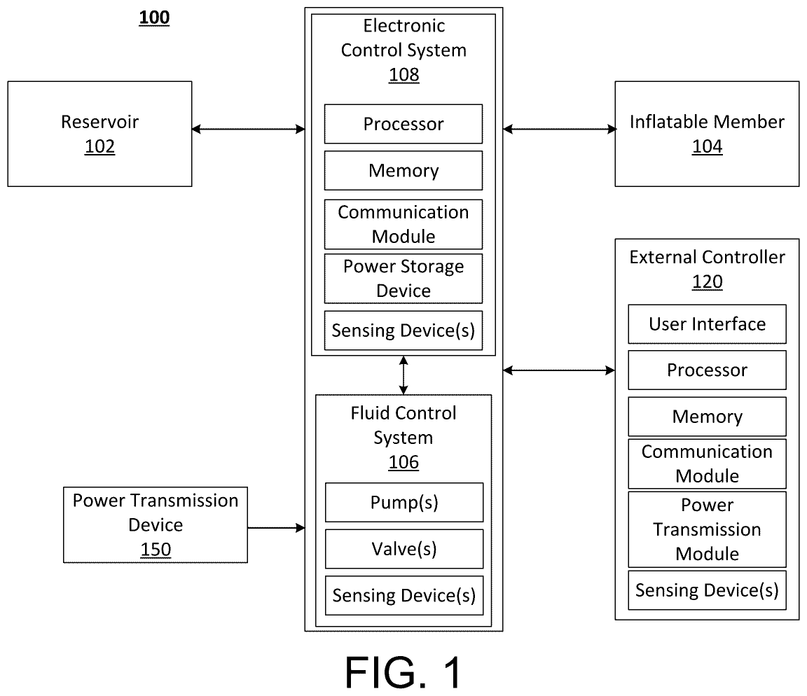

FIG. 1 is a block diagram of an example implantable fluid operated inflatable device 100. The example inflatable device 100 shown in FIG. 1 includes a fluid reservoir 102, an inflatable member 104, and an electronic control system 108. The electronic control system 108 may interface with a fluid control system 106. The fluid control system 106 can include fluidics components such as one or more pumps, one or more valves and the like configured to transfer fluid between the fluid reservoir 102 and the inflatable member 104. The fluid control system 106 can include one or more sensing devices that sense conditions such as, for example, fluid pressure, fluid flow rate and the like within the fluidics architecture of the inflatable device 100. In some implementations, the electronic control system 108 includes components that provide for the monitoring and/or control of the operation of various fluidics components of the fluid control system 106 and/or communication with one or more sensing device(s) within the implantable fluid operated inflatable device 100 and/or communication with one or more external device(s). In some examples, the electronic control system 108 includes components such as a processor, a memory, a communication module, a power storage device, or battery, sensing devices such as, for example an accelerometer, and other such components configured to provide for the operation and control of the implantable fluid operated inflatable device 100. In some examples, the communication module of the electronic control system 108 may provide for communication with one or more external devices such as, for example, an external controller 120.

In some examples, the external controller 120 includes components such as, for example, a user interface, a processor, a memory, a communication module, a power transmission module, and other such components providing for operation and control of the external controller 120 and communication with the electronic control system 108 of the inflatable device 100. For example, the memory may store instructions, applications and the like that are executable by the processor of the external controller 120. The external controller 120 may be configured to receive user inputs via, for example, the user interface, and to transmit the user inputs, for example, via the communication module, to the electronic control system 108 for processing, operation and control of the inflatable device 100. Similarly, the electronic control system 108 may, via the respective communication modules, transmit operational information to the external controller 120. This may allow operational status of the inflatable device 100 to be provided, for example, through the user interface of the external controller 120, to the user, may allow diagnostics information to be provided to a physician, and the like.

In some examples, the power transmission module of the external controller 120 provides for charging of the components of the internal electronic control system 108. In some examples, transmission of power for the charging of the internal electronic control system 108 can be, alternatively or additionally, provided by an external power transmission device 150 that is separate from the external controller 120. In some implementations the external controller 120 can include sensing devices such as one or more pressure sensors, one or more accelerometers, and other such sensing devices. In some implementations, a pressure sensor in the external controller 120 may provide, for example, a local atmospheric or working pressure to the internal electronic control system 108, to allow the inflatable device 100 to compensate for variations in pressure. In some implementations, an accelerometer in the external controller 120 may provide detected patient movement to the internal electronic control system 108 for control of the inflatable device 100.

The fluid reservoir 102, the inflatable member 104, the electronic control system 108 and the fluid control system 106 may be internally implanted into the body of the patient. In some implementations, the electronic control system 108 and the fluid control system 106 are coupled in or incorporated into a housing. In some implementations, at least a portion of the electronic control system 108 is physically separate from the fluid control system 106. In some implementations, some modules of the electronic control system 108 are coupled to or incorporated into the fluid control system 106, and some modules of the electronic control system 108 are separate from the fluid control system 106. For example, in some implementations, some modules of the electronic control system 108 are included in an external device (such as the external controller 120) that is in communication other modules of the electronic control system 108 included within the implantable device 100. In some implementations, at least some aspects of the operation of the implantable fluid operated inflatable device 100 may be manually controlled.

In some examples, electronic monitoring and control of the fluid operated inflatable device 100 may provide for improved patient control of the device, improved patient comfort, improved patient safety, and the like. In some examples, electronic monitoring and control of the fluid operated device 100 may afford the opportunity for tailoring of the operation of the inflatable device 100 by a physician without further surgical intervention. Fluidic architecture defining the flow and control of fluid through the fluid operated inflatable device 100, including the configuration and placement of fluidics components such as pumps, valves, sensing devices and the like, may allow the inflatable device 100 to precisely monitor and control operation of the inflatable device, effectively respond to user inputs, and quickly and effectively adapt to changing conditions both within the inflatable device 100 (changes in pressure, flow rate and the like) and external to the inflatable device 100 (pressure surges due to physical activity, impacts and the like, sustained pressure changes due to changes in atmospheric conditions, and other such changes in external conditions).

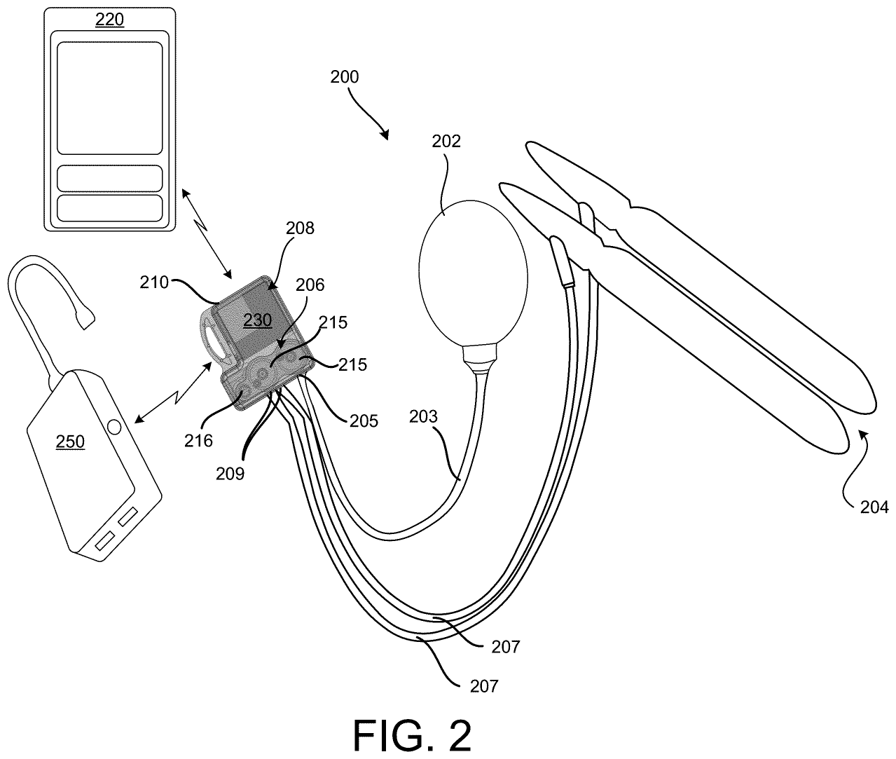

The example implantable fluid operated inflatable device 100 may be representative of a number of different types of implantable fluid operated devices. For example, the device 100 shown in FIG. 1 may be representative of an inflatable penile prosthesis as shown in FIG. 2. In some implementations, the example implantable fluid operated inflatable device 100 shown in FIG. 1 may be representative of other types of implantable inflatable devices that rely on the control of fluid flow to components of the device to achieve inflation, pressurization, deflation, depressurization, deactivation, and the like, such as, for example, an artificial urinary sphincter, and other such devices.

An example system including an example implantable fluid operated inflatable device 200 in the form of an example inflatable penile prosthesis is shown in FIG. 2. The example inflatable device 200 includes a fluid control system 206 (similar to the example fluid control system 106 described above with respect to FIG. 1) including fluidics components such as pumps, valves, sensing devices and the like positioned in fluid passageways. In some implementations, the fluid control system includes one or more fluid control devices 215, one or more pressure sensors 216, and other such components. The example inflatable device 200 includes an electronic control system 208 (similar to the example electronic control system 108 described above with respect to FIG. 1) configured to provide for the transfer of fluid between a reservoir 202 (such as the example reservoir 102 described above with respect to FIG. 1) and an inflatable member 204 (similar to the example inflatable member 104 described above with respect to FIG. 1) in the form of a pair of inflatable cylinders, via the fluidics components. Fluidics components of the fluid control system 206, and electronic components of the electronic control system 208 may be received in a housing 210. In some implementations, fluidics components of the fluid control system 206, and electronic components of the electronic control system 208 received in the housing 210 may together define an electronically controlled fluid manifold 230 that provides for the electronic control of the flow of fluid between the reservoir 202 and the inflatable member 204. A first conduit 203 connects a first fluid port 205 of the electronically controlled fluid manifold 230 (the fluid control system 206/electronic control system 208 received in the housing 210) with the reservoir 202. One or more second conduits 207 connect one or more second fluid ports 209 of the electronically controlled fluid manifold 230 (the fluid control system 206/electronic control system 208 received in the housing 210) with the inflatable member 204 in the form of the inflatable cylinders. The electronic control system 208 can communicate with an external controller 220 (similar to the external controller 120 described above with respect to FIG. 1), via respective communication modules. For example, an application stored in a memory and executed by a processor of the external controller 220 may allow the user and/or a physician to operate, view, monitor and alter operation of the inflatable device 200. In some examples, components of the electronic control system 208 and/or the fluid control system 206 may be charged and/or recharged by a power transmission module of the external controller 220, and/or by a power transmission device 250, that is separate from the external controller 220.

The principles to be described herein may be applied to the example implantable fluid operated inflatable device, in the form of the inflatable penile prostheses shown in FIG. 2, and other types of implantable fluid operated inflatable devices that rely on a pump assembly including various fluidics components to provide for the transfer of fluid between the different fluid filled implantable components to achieve inflation, deflation, pressurization, depressurization, deactivation, occlusion, and the like for effective operation. The example implantable fluid operated inflatable device 200 shown in FIG. 2 includes an electronic control system 208 to provide for control of the operation of the respective inflatable members 204 in the form of cylinders, and the monitoring and control of pressure and/or fluid flow through inflatable members 204. Some of the principles to be described herein may also be applied to implantable fluid operated inflatable devices that are manually controlled.

As noted above, the electronic control system 208 controlling the flow of fluid between the reservoir 202 and the inflatable member 204 for inflation, pressurization, deflation, depressurization and the like of the inflatable member 204 may provide for improved patient control of the inflatable device 200, improved accuracy in operation of the inflatable device 200, improved patient comfort, improved patient safety, and the like. However, in some situations, a size and/or a configuration of the electronic control system 208 and/or the fluid control system 206 (i.e., a size and/or a configuration of the electronically controlled fluid manifold 230 including the electronic control system 208 and the fluid control system 206) may pose a challenge for some patients. Accordingly, in some implementations, the electronically controlled fluid manifold 230 may include a fluid control system 206 having one or more combined pump and valve devices. The use of combined pump and valve devices may reduce a number of active components within the electronically controlled fluid manifold 230, thus reducing an overall size of the electronically controlled fluid manifold 230.

A fluid control system, in accordance with implementations described herein, can include a pump assembly including, for example, one or more pump and valve devices within a fluid circuit of the pump assembly to control the transfer fluid between the fluid reservoir and the inflatable member. In some examples, the pump assembly including the one or more pump and valve device(s) is electronically controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the one or more pump/valve device(s) include piezoelectric elements. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization, and deactivation of the components of the implantable fluid operated device to provide for patient safety and device efficacy.

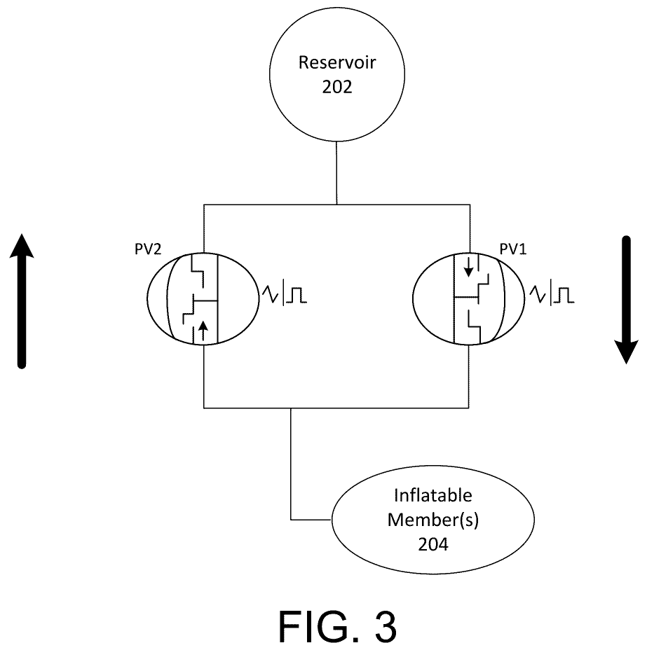

FIG. 3 is a schematic diagram of an example fluidic architecture for an implantable fluid operated inflatable device, according to an aspect. The fluidic architecture shown in FIG. 3 includes combination pump/valves positioned between the reservoir 202 and the inflatable member 204, to control the flow of fluid between the reservoir 202 and the inflatable member 204. The fluidic architecture of an implantable fluid operated inflatable device can include other arrangements of fluidic channels, pump(s)/valve(s), pressure sensor(s) and other components than shown in FIG. 3.

In particular, the example fluidic architecture shown in FIG. 3 includes a first fluid control device, or combined pump and valve device, PV1 positioned in a first fluid passageway and controlling the flow of fluid from the reservoir 202 to the inflatable member 204, and a second fluid control device, or combined pump and valve device, PV2 positioned in a second fluid passageway and controlling the flow of fluid from the inflatable member 204 to the reservoir 202.

In the example arrangement shown in FIG. 3, the first combined pump and valve device PV1 and the second combined pump and valve device PV2 may be operated in a first mode to inflate or pressurize the inflatable member 204, and in a second mode to deflate or repressurize the inflatable member 204. In the first mode of operation, the first combined pump and valve device PV1 may be operable to convey fluid from the reservoir 202 to the inflatable member 204, while the second combined pump and valve device PV2 remains closed/inoperable to prevent flow of fluid from the inflatable member 204 towards the reservoir 202 to prevent deflation/depressurization. The first combined pump and valve device PV1 may remain operable to pump fluid to the inflatable member 204 until a desired pressure is achieved. The first combined pump and valve device PV1 may be closed once the desired pressure is achieved, to maintain the inflatable member 204 at the desired pressure/inflated state. In the second mode of operation, the second combined pump and valve device PV2 may be operable to convey fluid from the inflatable member 204 to the reservoir 202, while the first combined pump and valve device PV1 remains closed/inoperable to prevent flow of fluid from the reservoir 202 towards the inflatable member 204 to prevent inflation/pressurization. The second combined pump and valve device PV2 may remain operable to pump fluid to the reservoir 202 until a desired pressure is achieved at the inflatable member 204. The second combined pump and valve device PV2 may be closed once the desired pressure is achieved, to maintain the inflatable member 204 at the desired pressure/in the deflated state.

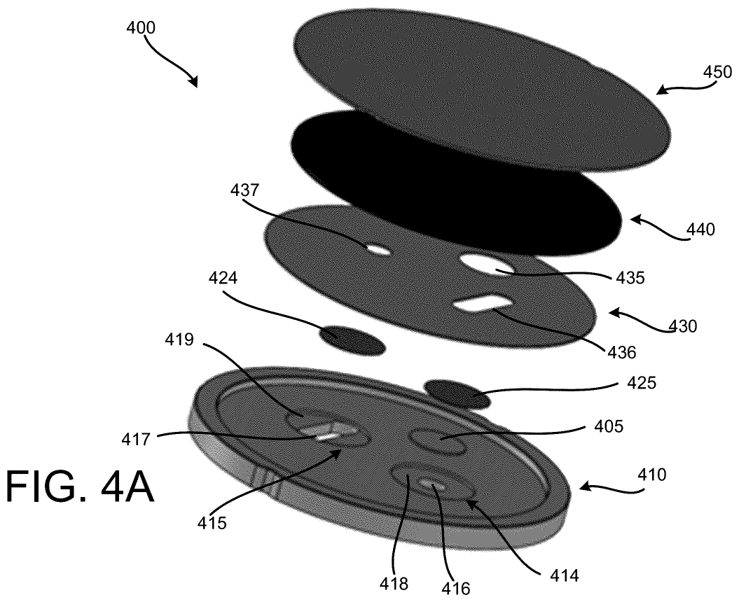

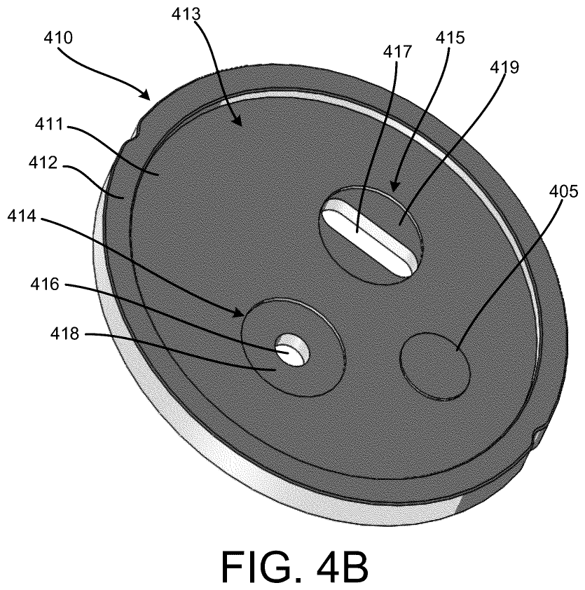

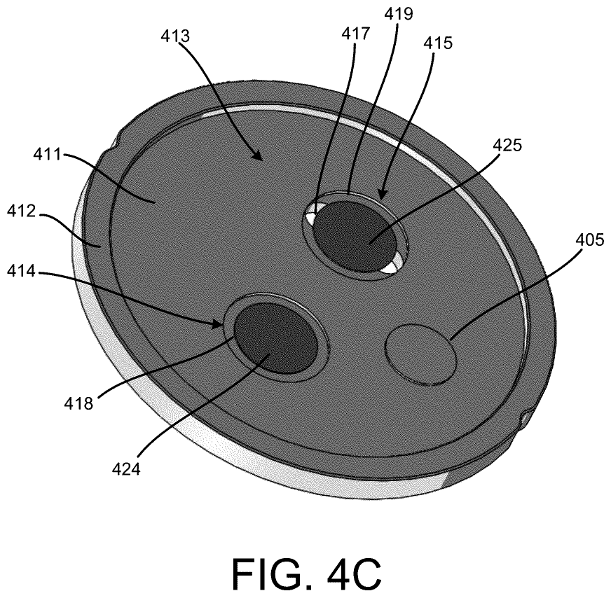

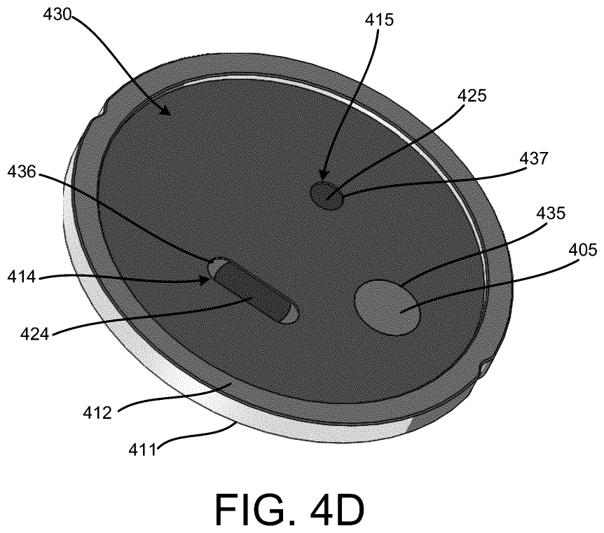

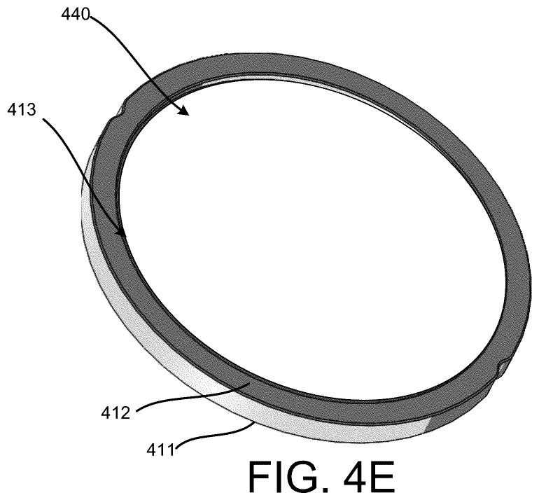

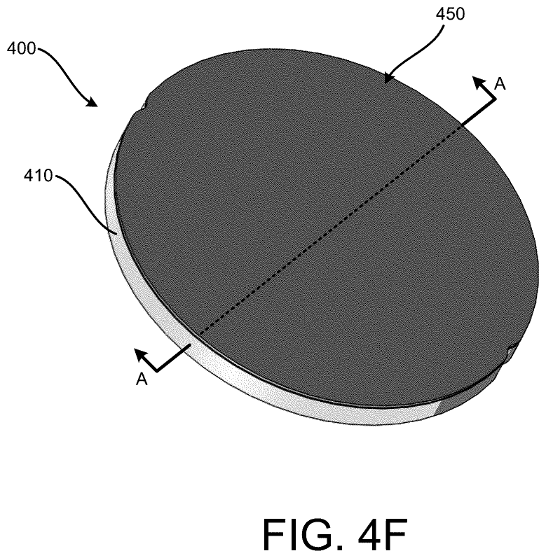

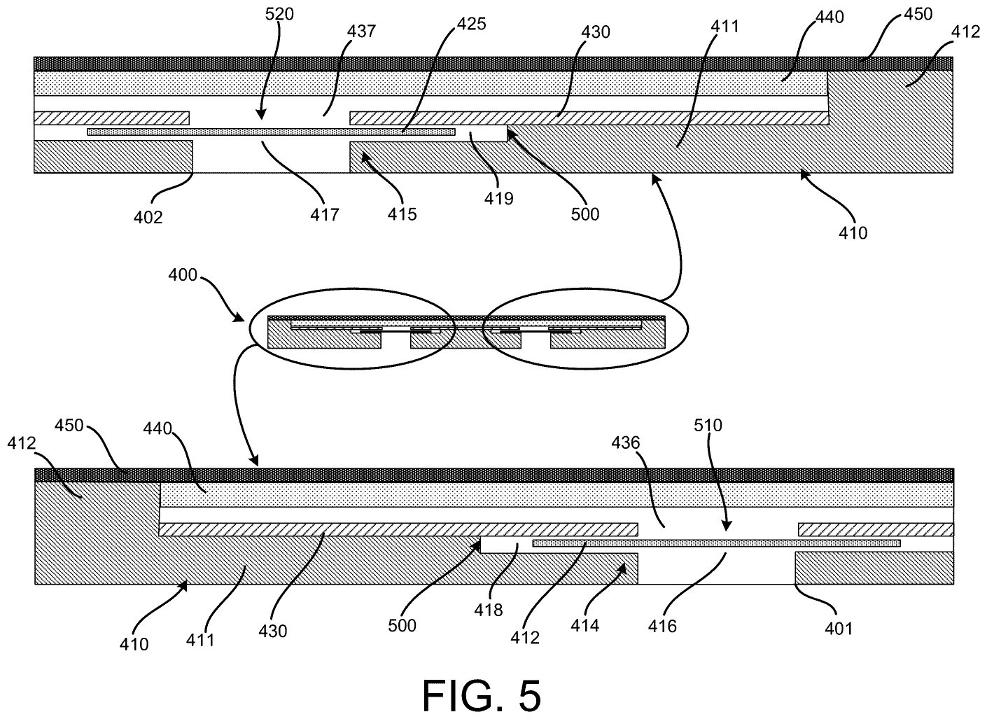

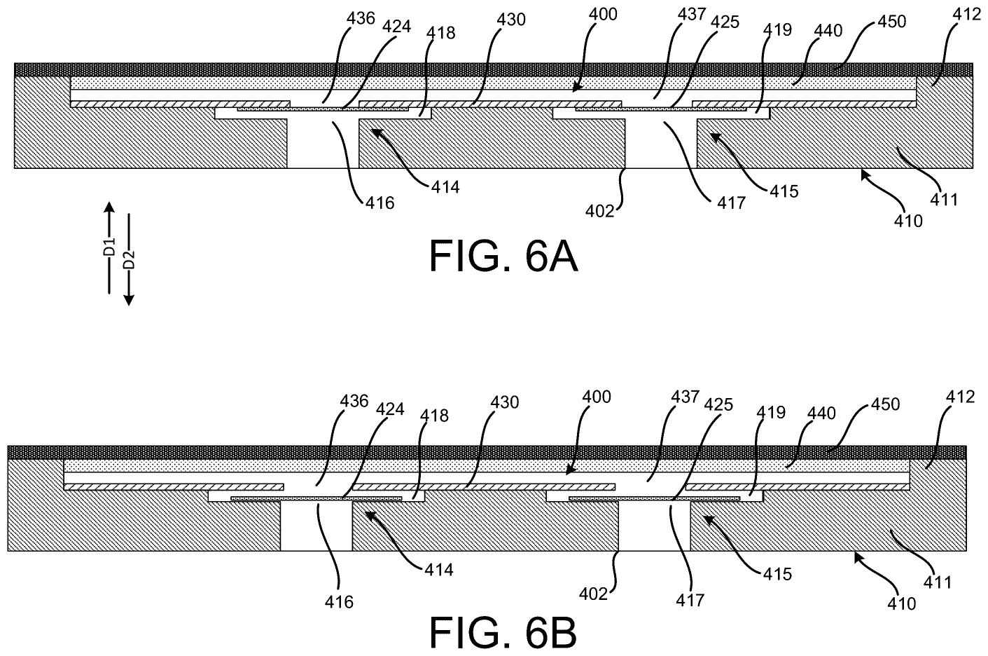

FIG. 4A is an exploded perspective view of an example combined pump and valve device 400, in accordance with implementations described herein. FIGS. 4B-4E illustrate a sequential assembly of the example combined pump and valve device 400. FIG. 4F is an assembled perspective view of the example combined pump and valve device 400. FIG. 5 is a cross-sectional view of the example combined pump and valve device 400, taken along line A-A of FIG. 4F. FIGS. 6A and 6B are cross-sectional views illustrating operation of the example combined pump and valve device 400. The example combined pump and valve device 400 shown in FIGS. 4A through 6B may be included in the fluid control system 206 of the example electronically controlled fluid manifold 230 described above. For example, the fluid control devices 215 of the electronically controlled fluid manifold 230 may be defined by the combined pump and valve device 400 to be described below with respect to FIGS. 4A through 6B. The first combined pump and valve device PV1 and/or the second combined pump and valve device PV2 shown in FIG. 3 may be defined by the combined pump and valve device 400 to be described below with respect to FIGS. 4A through 6B.

As shown in the example arrangement illustrated in FIG. 4A, the combined pump and valve device 400 may include a base plate 410 defining a base portion of the combined pump and valve device 400. As shown in FIG. 4B, the base plate 410 may include a base portion 411, with a rim portion 412 extending along a peripheral portion of the base portion 411. The base portion 411 and the rim portion 412 may define a recessed portion 413 in which components of the combined pump and valve device 400 are received. A depth of the recessed portion 413 formed in the base plate 410 may define a height of a fluid chamber in which fluid is held and through which fluid is pumped, and a corresponding pump stroke volume.

A first valve pocket 418 is defined in a first portion of the base portion 411. A first opening 416 is defined in the first valve pocket 418. The first opening 416 may be in fluid communication with a first fluid channel 401 (see FIGS. 6A and 6B) that guides fluid into the combined pump and valve device 400. In an example in which the combined pump and valve device 400 is included in the electronically controlled fluid manifold 230 of the inflatable device 200 described above, the first fluid channel 401 may be in fluid communication with the first conduit 203 that guides fluid from the reservoir 202 to the electronically controlled fluid manifold 230. In some implementations, the first valve pocket 418 including the first opening 416 may define a first port 414, for example, an inlet port, through which fluid may be introduced into the combined pump and valve device 400. A second valve pocket 419 is defined in a second portion of the base portion 411. A second opening 417 is formed in the second valve pocket 419. The second opening 417 may be in fluid communication with a second fluid channel 402 (see FIGS. 6A and 6B) that guides fluid out of the combined pump and valve device 400. In an example in which the combined pump and valve device 400 is included in the electronically controlled fluid manifold 230 of the inflatable device 200 described above, the second fluid channel 402 may be in fluid communication with the one or more second conduits 207 that guide fluid from the electronically controlled fluid manifold 230 to the inflatable member 204. In some implementations, the second valve pocket 419 including the second opening 417 may define a second port 415, for example, a discharge port, through which fluid is discharged from, or pumped from, the combined pump and valve device 400. In the example shown in FIG. 4B, the first (inlet) opening 416 is substantially circular, and the second (discharge) opening 417 has an oblong shape, or a substantially elliptical or oval shape. In some examples, an indexing device may be provided on the base plate 410. The indexing device may facilitate alignment and placement of a passive foil layer 430 (see FIG. 4D). In the example shown in FIG. 4B, an indexing device 405 in the form of a circular feature is provided on the base portion 411 of the base plate 410.

In some implementations, the base plate 410 may be formed of a material that is implantable in a patient, such as, for example, a titanium material or other such material. In some implementations, the base plate 410 may be machined into a surface of the housing 210 of the electronically controlled fluid manifold 230. In some implementations, the base plate 410 is fabricated as a separate unit or part, independent from the housing 210. This may allow the combined pump and valve device 400 to be assembled and tested prior to installation within the electronically controlled fluid manifold 230.

As shown in FIG. 4C, a first disc 424 may be positioned in the first valve pocket 418, and a second disc 425 is positioned in the second valve pocket 419. The first disc 424 may float in the first valve pocket 418. In the example shown in FIG. 4C, a contour of the first disc 424 corresponds to a contour of the first valve pocket 418, such that the first disc 424 is movable within the first valve pocket 418. A contour of the second disc 425 corresponds to a contour of the second valve pocket 419, such that the second disc 425 is movable within the second valve pocket 419. The first disc may selectively open and close the first opening 416 in the first valve pocket 418, such that the first disc 424 defines a functional element of a check valve at the first port 414. The first disc 424 extends fully across the first opening 416 to fully close the first opening 416 when the first disc 424 is positioned against the surface of the first valve pocket 418 during operation of the combined pump and valve device 400.

The second disc 425 may float in the second valve pocket 419. When the second disc is positioned against the second opening 417 formed in the second valve pocket 419 of the base plate 410, the second disc partially obstructs a portion of the second opening, leaving opposite end portions of the elliptically configured second opening 417 open and unobstructed, allowing fluid to flow therethrough. That is, in the example shown in FIG. 4C, end portions of the second opening 417 extend beyond the outer periphery of the second disc 425, such that the end portions of the second opening 417 remain open, even with the second disc 425 positioned against the surface of the second valve pocket 419 during operation of the combined pump and valve device 400.

As shown in FIG. 4D, a passive foil layer 430 is positioned in the recessed portion 413 of the base plate 410, such that the first disc 424 and the second disc 425 are positioned between the base plate 410 and the passive foil layer 430. In some implementations, the passive foil layer 430 is fixed to the base plate 410. In some implementations, an indexing device 435 provided on the passive foil layer 430 is indexed with the indexing device 405 provided on the base plate 410. A first opening 436 is formed in a first portion of the passive foil layer 430, corresponding to the position of the first disc 424 and/or the first port 414/first opening 416/first valve pocket 418 of the base plate 410. A second opening 437 is formed in a second portion of the passive foil layer 430, corresponding to the position of the second disc 425 and/or the second port 415/second opening 417/second valve pocket 419 of the base plate 410. In the example arrangement shown in FIG. 4D, the first opening 436 in the passive foil layer 430 has an oblong shape, or a substantially elliptical or oval shape. The second opening 417 in the passive foil layer 430 has a substantially circular shape. When the first disc 424 is positioned against the passive foil layer 430 during operation of the combined pump and valve device 400, end portions of the first opening 436 in the passive foil layer 430 remain open. When the second disc 425 is positioned against the passive foil layer 430 during operation of the combined pump and valve device 400, the second opening 417 is substantially fully blocked or closed by the second disc 425.

In some examples, an open area of the first opening 436 in the passive foil layer 430 extends beyond an outer periphery of the first disc 424, such that fluid can flow through the open end portions of the first opening 436 in the passive foil layer 430. In some examples, an open area of the second opening 417 in the base plate 410 extends beyond an outer periphery of the second disc 425, such that fluid can flow through the open end portions of the second opening 417 in the base plate 410.

As shown in FIG. 4F, an actuator plate 450 is coupled to the base plate 410, to secure the actuator plate 450 to the base plate 410. In some implementations, the actuator plate 450 may be made of a piezoelectric material. As shown in FIG. 4E, a seal 440 may be positioned on an interior facing side of the actuator plate 450. In some implementations, the seal 440 is coupled to, or bonded to, the interior facing side of the actuator plate 450. Adhering or bonding the seal 440 to the interior facing side of the actuator plate 450 may provide for positive positioning of the seal 440, reducing the risk of movement of the seal 440, thus reducing the risk of leakage. In some implementations, the seal 440 has a ring configuration. In some implementations, the seal 440 has a plate type configuration. The greater sealing surface area may further improve scaling integrity, thus improving functionality. Coupling of the actuator plate 450 to the base plate 410, for example, at the rim portion 412 of the base plate 410, forms an internal chamber 500 together with the recessed portion 413 of the base plate 410, and within the first and second valve pockets 418, 419, as illustrated in the cross-sectional views shown in FIGS. 6A and 6B. The chamber 500 has a specific volume that defines a volumetric flow rate of the pumping capacity of the combined pump and valve device 400.

In some implementations, the first port 414, the first opening 416, the first valve pocket 418, and the floating position of the first disc 424 positioned in the first valve pocket 418, together define a first fluid control device 510, in the form of a uni-directional pocket check valve that controls the flow of fluid into the chamber 500, based on a position of the first disc 424 relative to the first opening 416 formed in the base plate 410 and the first opening 436 formed in the passive foil layer 430. In some implementations, the second port 415, the floating position of the second disc 425 in the second valve pocket 419, and the second opening 417 together define a second fluid control device 520, in the form of a uni-directional pocket check valve that controls the flow of fluid out of the chamber 500, based on a position of the second disc 425 relative to the second opening 417 in the base plate 410 and the second opening 417 in the passive foil layer 430.

In some implementations, voltage may be applied to the actuator plate 450 to cause fluid to be drawn into the chamber 500 through the first fluid control device 510/first port 414, and discharged from the chamber 500 through the second fluid control device 520/second port 415. In some implementations, varying voltages may be applied to the actuator plate 450. The varying voltages may be selectively applied to cause movement of the first disc 424 and/or the second disc 425 in a desired direction within the respective valve pocket 418, 419, and to convey or restrict flow of fluid therethrough.

FIGS. 6A and 6B illustrate operation of the combined pump and valve device 400, in accordance with implementations described herein.

In some implementations, a first voltage and a second voltage may be applied to the actuator plate 450 to cause the combined pump and valve device 400 to pump fluid from the reservoir 202 to the inflatable member 204, for inflation of the inflatable member 204. The first voltage and the second voltage may be applied in an alternating manner to cause a filling action that draws fluid from the reservoir 202 into the combined pump and valve device 400, and a pumping action that pumps the fluid from the combined pump and valve device 400 to the inflatable member 204.

As shown in FIG. 6A, in response to application of the first voltage, the first disc (floating) disc 424 may be drawn upward (in the example orientation shown in FIG. 6A), in the direction of the arrow D1, and positioned against the surface of the passive foil layer 430, at a position corresponding to the first opening 436 in the passive foil layer 430. In response to application of the first voltage, the second (floating) disc 425 may be drawn upward (in the example orientation shown in FIG. 6A), in the direction of the arrow D1, and positioned against the surface of the passive foil layer 430, at a position corresponding to the second opening 437 in the passive foil layer 430. In some examples, the first voltage may be applied to a piezoelectric element (not shown in FIGS. 6A and 6B) coupled to the actuator plate 450, causing slight deformation or deflection (for example, in the direction of the arrow D1) that generates a suction force (for example, in the direction of the arrow D1), causing

the first disc 424 and the second disc 425 to be against the surface of the passive foil layer 430. This opens the first (inlet) port 414 and closes the second (exit) port 415, allowing the chamber 500 to be filled with fluid from the reservoir 202 through the first opening 416/first port 414. That is, with the first disc 424 positioned against the passive foil layer 430, fluid flows through the first (circular) opening 416 in the base plate 410, and through the exposed, open end portions of the first (elliptical) opening 436 in the passive foil layer 430. That is, as the exposed, open end portions of the first opening 436 in the passive foil layer 430 are not blocked by the first disc 424, fluid can flow into the chamber 500 through the first openings 416, 436. With the second disc 425 positioned against the passive foil layer 430, the second (circular) opening 437 in the passive foil layer 430 is blocked by the second disc 425. Thus, fluid cannot flow through the second port 415 with the second disc 425 positioned against the surface of the passive foil layer 430. The first disc 424 and the second disc 425 may remain in the position shown in FIG. 6A until the chamber 500 is filled, or until a set volume of fluid has been filled in the chamber 500.

Once the chamber 500 has been filled with fluid, the second voltage may be applied to the actuator plate 450. As shown in FIG. 6B, in response to application of the second voltage, the first disc (floating) disc 424 may be drawn downward (in the example orientation shown in FIG. 6B) and positioned against the surface of the first valve pocket 418 of the base plate 410. In some examples, the second voltage may be applied to a piezoelectric element (not shown in FIGS. 6A and 6B) coupled to the actuator plate 450. In some examples, application of the second voltage may allow the actuator plate 450/piezoelectric element to return to an at rest state from the deflected state generated by application of the first voltage. In some examples, the application of the second voltage may cause slight deformation or deflection, for example, beyond the at rest state, for example, in the direction of the arrow D2. In response to application of the second voltage, the second (floating) disc 425 may be drawn downward (in the example orientation shown in FIG. 6B) and positioned against the surface of the second valve pocket 419 of the base plate 410. The positioning of the first disc 424 against the surface of the first valve pocket 418 and the second disc 425 against the surface of the second valve pocket 419 closes the first (inlet) port 414 and opens the second (exit) port 415, allowing the fluid held in the chamber 500 to be discharged through the second port 415 and pumped to the inflatable member 204. With the first disc 424 positioned against first valve pocket 418, the first (circular) opening 416 in the first valve pocket 418 of the base plate 410 is blocked by the first disc 424. Thus, fluid cannot flow through (i.e., back flow through) the first port 414 with the first disc 424 positioned against the first opening 416 in the first valve pocket 418 of the base plate 410. With the second disc 425 positioned against the second (elliptical) opening 417 in the second valve pocket 419 of the base plate 410, fluid flows from the chamber 500, through the exposed, open end portions of the second (elliptical) opening 417 in the second valve pocket 419 of the base plate 410. That is, as the exposed, open end portions of the second opening 417 in the second valve pocket 419 of the base plate 410 are not blocked by the second disc 425, fluid can flow out of the chamber the chamber 500 through the second opening 417 in the base plate 410, and into the second fluid channel 402 for supply to the inflatable member 204.

In some implementations, the first voltage and the second voltage can continue to be alternately applied, or pulsed, to perform a fluid pumping action. In some implementations, the fluid pumping action pumps fluid from the reservoir 202 to the inflatable member 204 of the example inflatable device 200, until a desired inflation pressure has been achieved. In some implementations, these voltages may be applied to the actuator plate 450 at a set frequency to generate a desired volumetric flow rate in the process of inflating the inflatable member 204. Once the desired pressure has been achieved, the desired pressure can be maintained by positioning the second disc 425 against the surface of the passive foil layer 430 to close the second port 415, as described above. The desired pressure can also be maintained by positioning the first disc 424 against the first valve pocket 418 of the base plate 410 to close the first port 414 as described above.

In some implementations, the fluid pumping action pumps fluid from the inflatable member 204 to the reservoir 202 of the example inflatable device 200, for depressurization or deflation of the inflatable member 204. In this mode of operation, voltages may be applied in an alternating manner, or pulsed, causing fluid to be drawn into the chamber 500 from the inflatable member 204 and pumped out of the chamber 500 back to the reservoir 202 until a desired level of depressurization or deflation is achieved.

In some implementations, a magnitude and a frequency of the alternating voltages applied to the actuator plate 450 may be based on a desired volumetric flow rate of fluid through the combined pump and valve device 400. In some implementations, the desired volumetric flow rate may be based on a desired inflation and/or deflation rate to be achieved, a fluid capacity of the combined pump and valve device 400, and other such factors. In some implementations, the voltage applied to the actuator plate 450 may be in a range of voltages.

The combined pump and valve device 400 described above with respect to FIGS. 1 through 6B incorporates pocket check valves in the fluid control system, to reduce an overall size of an electronically controlled fluid manifold, and to provide for electronically controlled inflation/pressurization and deflation/depressurization of an inflatable device of an implantable fluid operated inflatable device. In some implementations, an electronically controlled fluid manifold of an implantable fluid operated inflatable device may incorporate spring check valves.

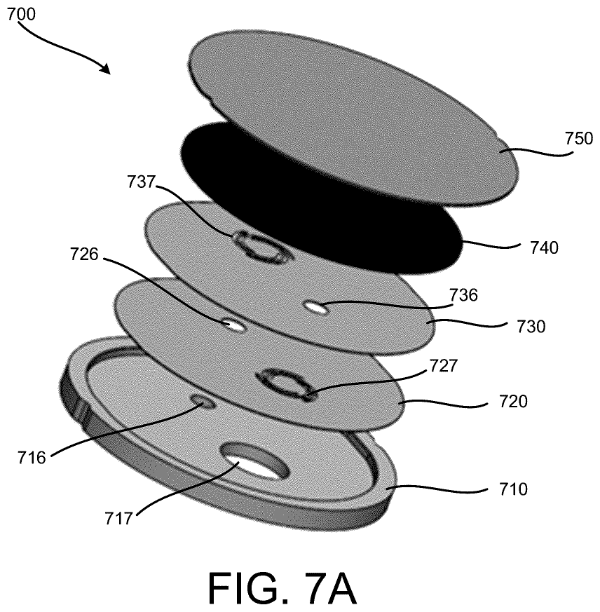

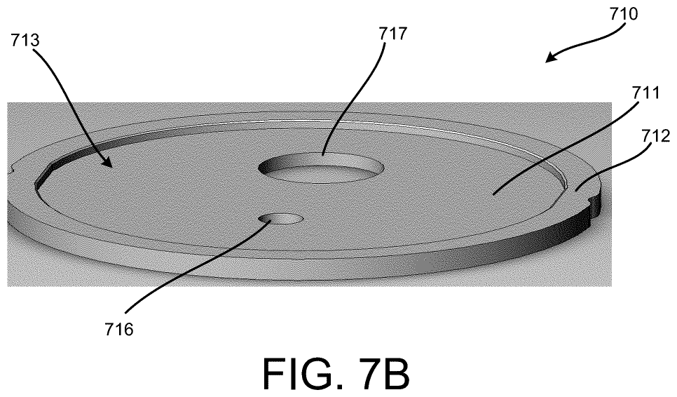

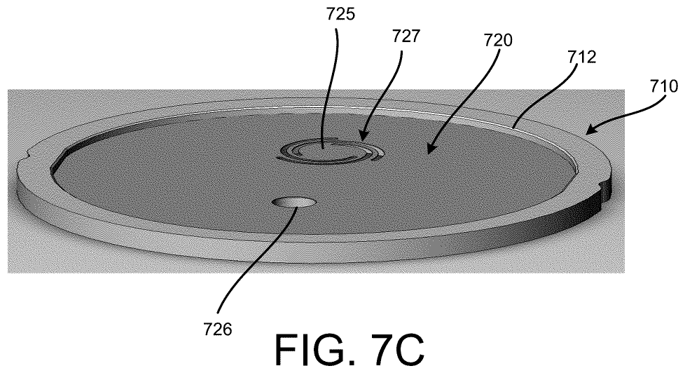

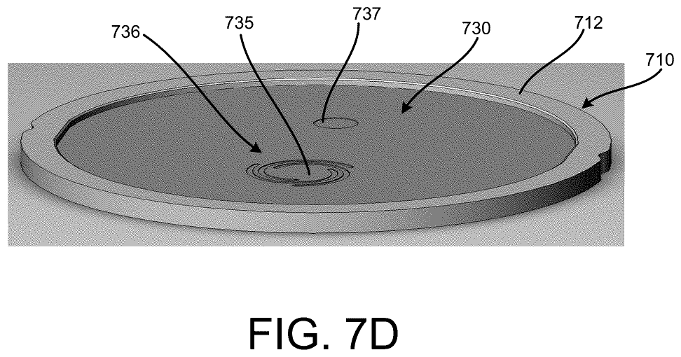

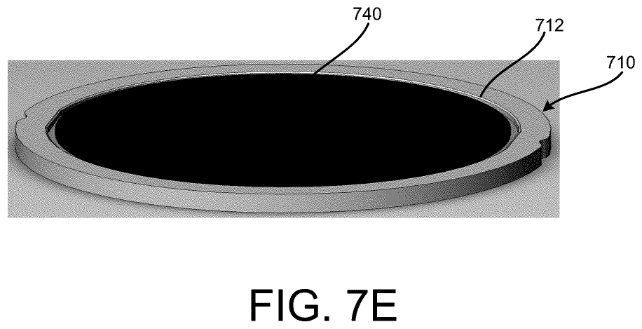

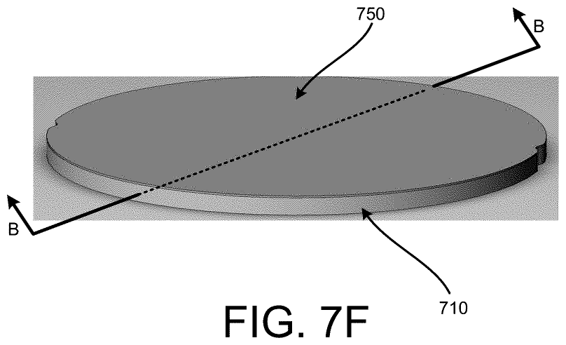

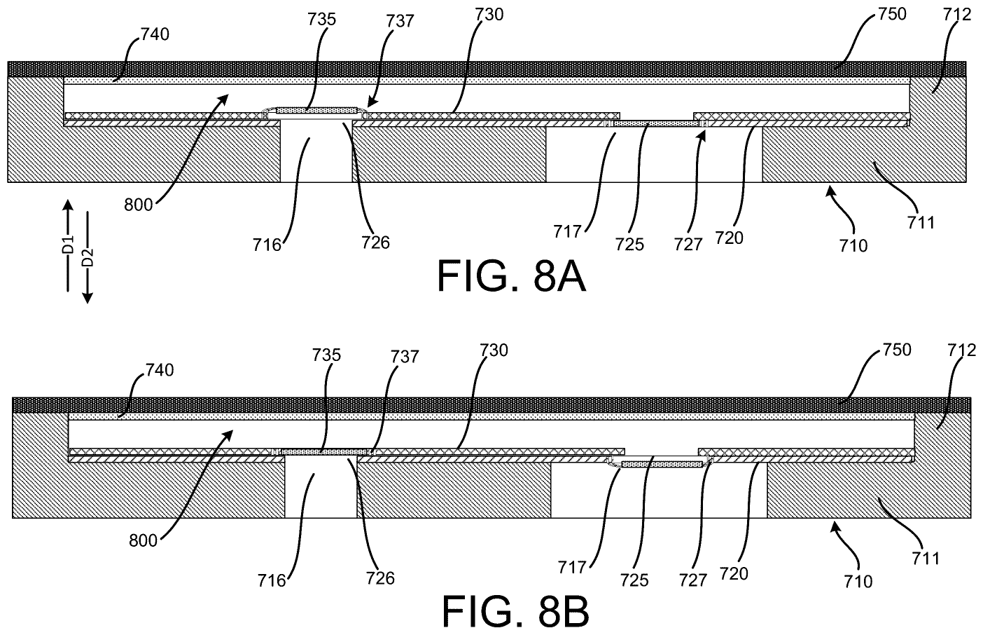

FIGS. 7A-7F and 8A-8B illustrate an example combined pump and valve device 700 including example spring check valves, that can be incorporated into an electronically controlled fluid manifold as described above. In particular, FIG. 7A is an exploded perspective view of the example combined pump and valve device 700. FIGS. 7B-7F illustrate a sequential assembly of the example combined pump and valve device 700. FIGS. 8A and 8B are cross-sectional views of the example combined pump and valve device 700, taken along line B-B of FIG. 7F.

As shown in the example arrangement illustrated in FIG. 7A, the combined pump and valve device 700 may include a base plate 710 defining a base portion of the combined pump and valve device 700. As shown in FIG. 7B, the base plate 710 may include a base portion 711, with a rim portion 712 extending along a peripheral portion of the base portion 711. A first opening 716 and a second opening 717 are formed in the base portion 711 of the base plate 710. In the example shown in FIG. 7B, an area of the second opening 717 is greater than an area of the first opening. The base portion 711 and the rim portion 712 may define a recessed portion 713 in which components of the combined pump and valve device 700 are received. A depth of the recessed portion 713 formed in the base plate 710 may define a height of a fluid chamber in which fluid is held and through which fluid is pumped, and a corresponding pump stroke volume.

A first passive foil layer 720 may be positioned in the recessed portion 713 of the base plate 710. In some examples, the first passive foil layer 720 is fixed to the base plate 710. As shown in FIG. 7C, the first passive foil layer 720 may include an opening 726. The opening may be formed in the first passive foil layer 720 at a position corresponding to the first opening 716 in the base plate 710. The first passive foil layer 720 may include a plurality of slots 727. The plurality of slots 727 may be formed at a position in the first passive foil layer 720 corresponding to the second opening 717 in the base plate 710. The plurality of slots 727 may provide for selective movement of a first disc 725 positioned at a central portion of the plurality of slots 727.

A second passive foil layer 730 may be positioned on the first passive foil layer 720. In some examples, the second passive foil layer 730 is fixed to the first passive foil layer 720, and to the base plate 710. As shown in FIG. 7D, the second passive foil layer 730 may include a plurality of slots 737. The plurality of slots 737 may be formed at a position corresponding the opening 726 in the first passive foil layer 720 and the first opening 716 formed in the base plate 710. The plurality of slots 737 may provide for selective movement of a second disc 735 positioned at a central portion of the plurality of slots 737 . . . . The second passive foil layer 730 may include an opening 736 formed at a position corresponding to the first disc 725 and the plurality of slots 727 formed in the first passive foil layer 720, and the second opening 717 in the base plate 710.

As shown in FIGS. 7E and 7F, a seal 740 may be positioned between the second passive foil layer 730 and an actuator plate 750. In some examples, the actuator plate 750 is fixed to the base plate 710. For example, the actuator plate 750 may be fixed to the rim portion 712 of the base plate 710. In some implementations, the seal 740 may be fixed to, or bonded to, or adhered to an interior facing side of the actuator plate 750.

As shown in FIG. 8A, in some implementations, a first voltage applied to the actuator plate 750 may cause a deflection, or deformation of the actuator plate 750 that generates a pressure, or suction force, for example, in the direction of the arrow D1. This force may draw the disc 735 of the spring check valve formed on the second passive foil layer 730 upward (in the example orientation shown in FIG. 8A), as shown in FIG. 8A. In this arrangement, the first opening 716 in the base plate 710 and the opening 726 in the first passive foil layer 720 are open, allowing fluid to flow into the chamber 800 through open areas defined by the plurality of slots 737extending from the second disc 735 in the extended position shown in FIG. 8A. In response to the application of the first voltage, movement of the first disc 725 in the direction of the arrow D1 is restricted by the second passive foil layer 730 at the opening 736. That is, a dimension, for example, a diameter of the first disc 725 may be greater than a corresponding dimension, for example, a diameter of the opening 736, so that the first disc 725 blocks the opening 736 in the position shown in FIG. 8A. Thus, in this arrangement, the second opening 717 is closed by the corresponding portion of the first passive foil layer 720, so that fluid is retained in the chamber 800.

In response to a second voltage applied to the actuator plate 750, the first disc 725 of the first passive foil layer 720 may be drawn downward (in the example orientation shown in FIG. 8B), as shown in FIG. 8B. In this arrangement, the second opening 717 in the base plate is no longer blocked, allowing fluid to flow out of the chamber 800 through open areas defined by the plurality of slots 727 extending from the first disc 725 in the extended position shown in FIG. 8B. In response to the application of the second voltage, movement of the second disc 735 in the direction of the arrow D2 is restricted by the first passive foil layer 730 at the opening 726. That is, a dimension, for example, a diameter of the second disc 735 may be greater than a corresponding dimension, for example, a diameter of the opening 726, so that the second disc 735 blocks the opening 726 in the position shown in FIG. 8B.

In some examples, the first voltage and/or the second voltage may be applied to a piezoelectric element (not shown in FIGS. 8A and 8B) coupled to the actuator plate 750. The first voltage and the second voltage may be alternately applied to perform a pumping action through the combined pump and valve device 700. In a first mode, the pumping action of the combined pump and valve device 700 may pump fluid from the reservoir 202 to the inflatable member 204 to inflate or pressurize the inflatable member 204. In a second mode of operation, the pumping action of the combined pump and valve device 700 may pump fluid from the inflatable member 204 to the reservoir 202, to deflate or depressurize the inflatable member 204.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable fluid operated inflatable device, comprising:
a fluid reservoir;
an inflatable member; and
a fluid control system configured to control fluid flow between the fluid reservoir and the inflatable member, the fluid control system including:
at least one combined pump and valve device positioned in one or more fluid passageways defined within a housing, the at least one combined pump and valve device including:
a base plate;
a passive foil layer positioned in a recessed portion of the base plate;
an actuator plate coupled to the base plate so as to define a chamber with the recessed portion of the base plate;
a first disc selectively engaging a first opening formed in a first valve pocket in the base plate or a first opening in the passive foil layer in response to a voltage applied to the actuator plate; and
a second disc selectively engaging a second opening formed in a second valve pocket in the base plate or a second opening in the passive foil layer in response to a voltage applied to the actuator plate.

2. The implantable fluid operated inflatable device of claim 1, wherein
the first opening in the base plate has a substantially circular shape,
the first opening in the passive foil layer has a substantially elliptical shape,
the second opening in the base plate has a substantially elliptical shape, and
the second opening in the passive foil layer has a substantially circular shape.

3. The implantable fluid operated inflatable device of claim 1, wherein
in response to a first voltage applied to the actuator plate, the first disc is positioned against the first opening in the passive foil layer, and the second disc is positioned against the second opening in the passive foil layer, and
in response to a second voltage applied to the actuator plate, the first disc is positioned against the first opening in the base plate, and the second disc is positioned against the second opening in the base plate.

4. The implantable fluid operated inflatable device of claim 3, wherein, in response to the first voltage applied to the actuator plate,
the first disc partially blocks the first opening in the passive foil layer; and
the second disc fully blocks the second opening in the passive foil layer.

5. The implantable fluid operated inflatable device of claim 4, wherein, in response to the first voltage applied to the actuator plate,
fluid flows into the chamber through the first opening formed in the base plate and through open end portions of the first opening in the passive foil layer; and
fluid is retained in the chamber by the second disc positioned against the second opening in the passive foil layer to fill the chamber with fluid.

6. The implantable fluid operated inflatable device of claim 5, wherein, in response to the second voltage applied to the actuator plate,
the first disc fully blocks the first opening in the base plate; and
the second disc partially blocks the second opening in the base plate.

7. The implantable fluid operated inflatable device of claim 6, wherein, in response to the second voltage applied to the actuator plate,
fluid flows out of the chamber through open end portions of the second opening formed in the passive foil layer and through the second opening in the base plate; and
backflow of fluid from the chamber is restricted by the first disc positioned against the first opening in the base plate.

8. The implantable fluid operated inflatable device of claim 1, wherein the base plate includes:
a base portion; and
a rim portion extending along a peripheral portion of the base portion,
wherein the recessed portion of the base plate is formed within an area defined by the base portion and the rim portion, and
wherein the first valve pocket and the second valve pocket are formed in the base portion of the base plate.

9. The implantable fluid operated inflatable device of claim 1, wherein the at least one combined pump and valve device includes a seal provided on an interior facing side of the actuator plate, wherein the seal extends into the recessed portion of the base plate to seal the chamber.

10. The implantable fluid operated inflatable device of claim 1, wherein
the first disc is a floating disc positioned within the first valve pocket formed in the base plate, such that the first disc is movable in the first valve pocket, between the first opening in the base plate and the first opening in the passive foil layer, in response to an application of voltage to the actuator plate; and
the second disc is a floating disc positioned within the second valve pocket formed in the base plate, such that the second disc is movable in the second valve pocket, between the second opening in the base plate and the second opening in the passive foil layer, in response to an application of voltage to the actuator plate.

11. The implantable fluid operated inflatable device of claim 1, wherein the actuator plate is operable in response to an application of voltage thereto by an electronic control system of the fluid control system.

12. The implantable fluid operated inflatable device of claim 11, wherein the actuator plate comprises a piezoelectric actuator.

13. The implantable fluid operated inflatable device of claim 1, wherein
the first valve pocket, the first opening in the base plate, the first disc, and the first opening in the passive foil layer define a first pocket valve controlling a flow of fluid from a first fluid passageway into the chamber; and
the second valve pocket, the second opening in the base plate, the second disc, and the second opening in the passive foil layer define a second pocket valve controlling a flow of fluid out of the chamber to at least one second fluid passageway.

14. The implantable fluid operated inflatable device of claim 13, wherein
the first fluid passageway provides for fluid communication between the fluid reservoir and the fluid control system, and
the at least one second fluid passageway provides for fluid communication between inflatable member and the fluid control system.

15. The implantable fluid operated inflatable device of claim 1, wherein the base plate is machined into a surface of the housing of the fluid control system, or fabricated as a separate unit from the housing of the fluid control system.

16. A fluid control system for an implantable fluid operated inflatable device, comprising:
a housing;
fluidic architecture defining one or more fluid passageways within the housing; and
at least one combined pump and valve device positioned a fluid passageway of the fluid control system, the at least one combined pump and valve device including:
a base plate;
an actuator plate coupled to a rim portion of the base plate so as to define a chamber with a recessed portion of the base plate;
a passive foil layer positioned between the base plate and the actuator plate;
a first disc selectively engaging a first opening formed in a first valve pocket in the base plate or a first opening in the passive foil layer in response to a voltage applied to the actuator plate; and
a second disc selectively engaging a second opening formed in a second valve pocket in the base plate or a second opening in the passive foil layer in response to a voltage applied to the actuator plate, wherein
an open area of the first opening in the passive foil layer extends beyond an outer periphery of the first disc, and
an open area of the second opening in the base plate extends beyond an outer periphery of the second disc.

17. The fluid control system of claim 16, wherein
in response to a first voltage applied to the actuator plate, the first disc is positioned against the first opening in the passive foil layer, and the second disc is positioned against the second opening in the passive foil layer, and
in response to a second voltage applied to the actuator plate, the first disc is positioned against the first opening in the base plate, and the second disc is positioned against the second opening in the base plate.

18. The fluid control system of claim 17, wherein, in response to the first voltage applied to the actuator plate,
the first disc partially blocks the first opening in the passive foil layer, allowing fluid to into the chamber through the first opening formed in the base plate and through open end portions of the first opening in the passive foil layer; and
the second disc fully blocks the second opening in the passive foil layer, retaining fluid to fill the chamber with fluid.

19. The fluid control system of claim 18, wherein, in response to the second voltage applied to the actuator plate,
the second disc partially blocks the second opening in the base plate, allowing fluid to flow out of the chamber through open end portions of the second opening formed in the passive foil layer and through the second opening in the base plate; and
the first disc fully blocks the first opening in the base plate, restricting backflow of fluid from the chamber.

20. The fluid control system of claim 19, further comprising a seal provided on an interior facing side of the actuator plate, wherein the seal extends into the chamber to seal the chamber.
